(19) United States
(12) Patent Application Publication
(10) Pub. No.: US 2019/0150840 A1
(43) Pub. Date: May 23, 2019

(54) TRAUMATIC BRAIN INJURY CATHETER

(71) Applicants: David Darrow, Minneapolis, MN (US); Samuel Cramer, Blaine, MN (US); Sean Lester Moen, Saint Paul, MN (US); Andrew Grande, Mendota Heights, MN (US)

(72) Inventors: David Darrow, Minneapolis, MN (US); Samuel Cramer, Blaine, MN (US); Sean Lester Moen, Saint Paul, MN (US); Andrew Grande, Mendota Heights, MN (US)

(21) Appl. No.: 16/198,351

(22) Filed: Nov. 21, 2018

Related U.S. Application Data
(60) Provisional application No. 62/590,027, filed on Nov. 22, 2017.

(51) Int. Cl. A61B 5/00; A61B 5/0478; A61B 5/03; A61N 1/05; A61M 27/00

(52) U.S. Cl. CPC A61B 5/6852; A61B 5/0478; A61B 5/031; A61M 2230/10; A61M 27/002; A61M 2210/0693; A61M 2205/3344; A61N 1/0529

(57) ABSTRACT

An extraventricular drain system can include a catheter. The catheter can have a shaft including a proximal end and a distal end, a lumen extended along a length of the catheter, and a drain located on the shaft. The drain can be fluidly coupled with the lumen. In an example, an electrode can be located along the shaft at an axial location. A reference contact can be located on the shaft at an axial location further from the distal end than the electrode. The electrode and the reference contact can be configured for electrical communication with a processing unit. In a further example, a first pressure port and a second pressure port can be located at the distal end and have different respective axial locations and respective radial locations. The first and second pressure ports can be in fluid communication with a first and second pressure sensors respectively.

### CROSS-REFERENCE TO RELATED APPLICATION

This patent application claims the benefit of priority to U.S. Provisional Application Ser. No. 62/590,027, entitled “TRAUMATIC BRAIN INJURY CATHETER,” filed Nov. 22, 2017, which is incorporated by reference herein in its entirety.

### TECHNICAL FIELD

This document pertains generally, but not by way of limitation, to catheters for placement in the brain.

### BACKGROUND

Traumatic brain injury (TBI) can have various causes, severities, and pathophysiology. In young people. TBI is the leading cause of morbidity and mortality in developed countries. TBI is a frequent result of accidental injury in the USA with approximately 2.5 million people affected per year, approximately 10% of whom requiring extended hospitalization, often in an intensive care unit. Around 275.000 (15.1%) of hospital admissions and 52,000 deaths in the USA each year are due to TBI. Those that survive the initial insult invariably face prolonged stays in a neurologic ICU, possible neurosurgical intervention, and prolonged period of post-acute supportive care. In the US, it is estimated that 5.3 million individuals are living with long-term disability as a result of TBI.

Given the complexity and duration of medical care that accompanies severe TBI, the cost of care associated with TBI are immense. It is estimated that total hospital charges for TBI-related admissions in 2010 alone were $21.4 billion. Beyond hospital charges, it is estimated that TBI costs the US economy $76.5 billion annually with the costs for disability and lost productivity outweighing those of acute medical care and rehabilitation. Some TBIs can result in permanent tissue damage and irreversible loss of brain function. Accordingly, options for therapeutic treatment can be limited.

Furthermore, secondary injury after severe TBI is thought to significantly increase the severity of the initial injury. Cortical Spreading Depression (CSD) is a form of secondary brain injury. CSD includes electrophysiological waves that depolarize neurons and astrocytes and disrupt local cortical function for minutes to hours, have been demonstrated to occur in severe TBI patients. CSD is associated with worse outcomes in patients after acute brain injuries such as TBI. To date, Electrocorticography (ECoG) monitoring of over 500 patients after TBI has shown that CSDs occur in 55-90% of individuals for days to weeks after the initial brain injury. These studies have demonstrated an initial peak in CSD frequency at 1-2 days after the TBI and a second peak at 6-7 days. Furthermore. CSD is associated with negative outcomes after TBI. Since a relationship between cortical spreading depression (CSD) events and worse outcomes after severe TBI have been demonstrated, a more thorough understanding of the factors that influence the initiation and frequency of CSDs after TBI is sought for the development of therapeutic strategies to reduce or block these secondary injury events from occurring.

Further secondary brain injuries can be associated with a subarachnoid hemorrhage (SAH) or ischemic stroke. SAH can be characterized by bleeding within the subarachnoid space of the brain. This can be correlated with a higher risk of stroke or seizure. One indication of SAH or stroke can be increased pressure within one or more ventricles of the brain.

Seizures (synchronous neuronal activity), such as post-traumatic seizures (PTS) can also result from TBIs. PTSs can correspond to more severe TBI and can also cause secondary brain injury. For instance, PTS can have various effects on the brain, such as reduce the oxygen available to the brain, cause excessive release of neurotransmitters, as well as raise the intercranial pressure among other things. Neuroimaging, such as MRI and CT scans are sometimes used to observe PTS symptoms and guide treatment.

In view of the above, there is a substantial need to diagnose and treat secondary brain injuries. Accordingly, devices and methods to detect and attenuate secondary brain injury are desirable.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of an extraventricular drain system including a plurality of pressure ports, according to an embodiment.

FIG. 2 is an example of an extraventricular drain system including a first pressure port having an axial location and a second pressure port having a radial location, according to an embodiment.

FIG. 3 is a block diagram of an example of a technique for measuring intercranial pressure, according to an embodiment.

FIG. 4 depicts an example of an extraventricular drain including a reference contact and an electrode located along a shaft of a catheter, according to an embodiment.

FIG. 5 is a block diagram of an example of a technique for measuring electrophysiological signals, according to an embodiment.

FIG. 6 is an example of an extraventricular drain including a plurality of electrodes having different radial locations along a shaft of a catheter, according to an embodiment.

FIG. 7 is a block diagram of an example of a technique for determining an origin of an electrophysiological signal using an extraventricular drain, according to an embodiment.

FIG. 8 illustrates a system level diagram in accordance with some embodiments of the invention.

### DETAILED DESCRIPTION

The present application relates to devices and techniques for an extraventricular catheter, such as an extraventricular catheter having a plurality of electrodes for measuring electrophysiological signals or a plurality of pressure ports for measuring intercranial pressure. The following detailed description and examples are illustrative of the subject matter disclosed herein; however, the subject matter disclosed is not limited to the following description and examples provided. Portions and features of some embodiments may be included in, or substituted for, those of other embodiments. Embodiments set forth in the claims encompass all available equivalents of those claims.

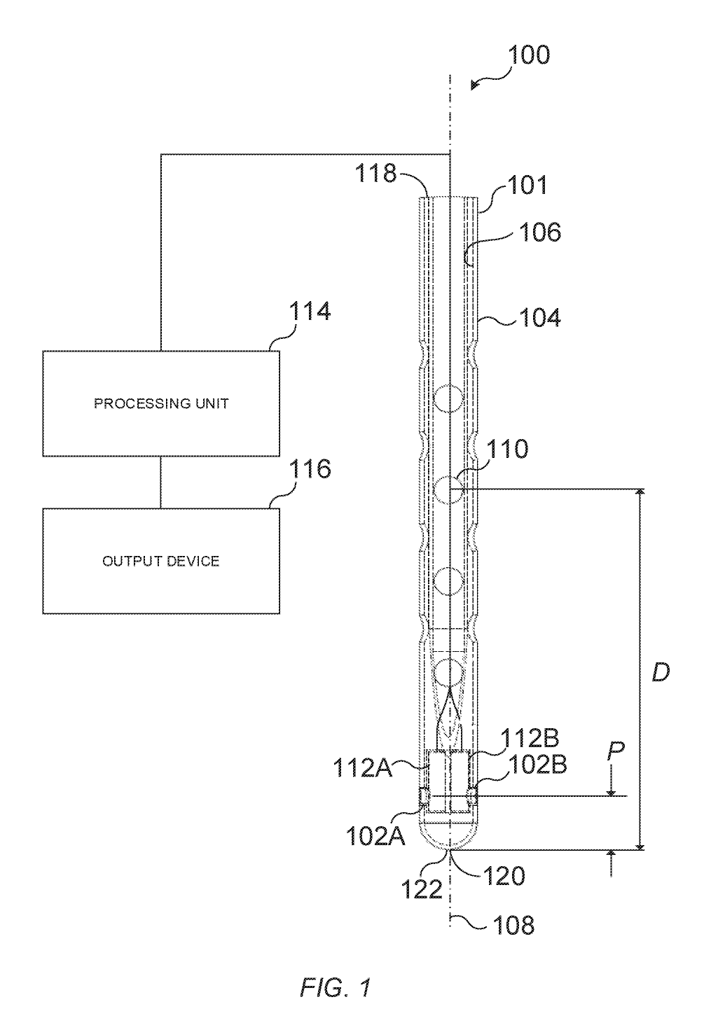

FIG. 1 illustrates an example of an extraventricular drain system 100 including a pressure port 102A (first pressure port) and a pressure port 102B (second pressure port). In an example, the extraventricular drain system 100 can include a catheter 101 having a shaft 104. The shaft 104 can include a lumen 106 extended along a length of the shaft 104, for instance, along a longitudinal axis 108 of the shaft 104. At least one drain 110 can be located along the shaft 104. The drain 110 can be fluidly coupled with the lumen 106, as shown in FIG. 1. The pressure port, such as the pressure port 102A and the pressure port 102B, can be configured for fluid communication with at least one pressure sensor, for instance, a pressure sensor 112A and a pressure sensor 112B in the example of FIG. 1. The one or more pressure ports can be configured for fluid communication with the respective pressure sensors. In some examples, the extraventricular drain system 100 can include a processing unit 114 and an output device 116. The processing unit 114 can be communicatively coupled to the pressure sensors, such as the first pressure sensor 112A and the second pressure sensor 112B. The processing unit 114 can receive a measured pressure from one or more of the pressure sensors and communicate a measured pressure to the output device 116.

The shaft 104 can include a proximal end 118 and a distal end 120. The distal end 120 can include an insertion tip 122 configured for insertion into a patient's body, such as a human brain. The distal end 120 opposes the proximal end 118 and includes a portion of a length of the shaft 104, such as the locations along the shaft 104 including the pressure ports 102A, 102B or the drain 110. In some examples, the shaft 104 can be fabricated from a material including, but not limited to, a biocompatible polymer or metal such as Polyether Ether Keytone (PEEK), Polyoxymethylene (POM), Polyphenylsulfone (PPSU), polypropylene, stainless steel, or titanium. The lumen 106 can extend along the length of the catheter 101 and through the proximal end 118. A first end of the lumen 106 can be open at the proximal end 118 and the second end of the lumen 106 can be located between the proximal end 118 and the distal end 120.

The drain 110 can be located on the shaft 104. In an example, the drain 110 can be located at a radial or an axial location along the shaft 104, or a combination thereof. The drain 110 can be fluidly coupled with the lumen 106. When inserted into a patient, such as within one or more ventricles of the brain, a pressure within the skull (e.g., ventricles of the brain) can be regulated by transferring cerebral spinal fluid (CSF) though the catheter 101 to reduce or increase the volume of CSF within the brain or skull, and correspondingly increase or reduce the pressure within the brain or skull. For instance, the CSF can be communicated through the drain 110 and along the lumen 106 of the catheter 101.

As discussed herein, the radial location can be a location on the shaft 104 at least partially transverse to the longitudinal axis 108. For instance, the radial location can be a location on the shaft 104 at a radial of the longitudinal axis 108 between 0 and 360 degrees. The radial location can be measured along a plane that is transverse to the longitudinal axis. For instance, the radial location can be an angular difference between features of the catheter 101 as measured perpendicular to the longitudinal axis 108. The axial location can be a location on the shaft 104 that is along the longitudinal axis 108, such as at the insertion tip 122 or at a location along the length of the shaft 104. In some examples, features of the catheter 101, such as the drain 110, pressure port 102A, or pressure port 102B, can include an axial location, a radial location, or a combination thereof. For instance, the various features can be located along the longitudinal axis 108 and therefore not have a radial location. In various examples, the drain 110 or pressure ports can include a radial location and an axial location. For instance, the drain 110 can be located on a first side (e.g., front or zero-degree radial) of the shaft 104 and at a distance D from the distal end 120.

In the example of FIG. 1, the catheter 101 includes two pressure ports, pressure port 102A and 102B. The one or more pressure ports, such as the pressure port 102A or the pressure port 102B, can be located at the distal end of the catheter shaft 104. The pressure port 102A can include a first axial location, a first radial location, or a combination thereof. The pressure port 102B can include a second axial location, a second radial location, or a combination thereof. The axial location can be a location on the shaft 104 along the longitudinal axis 108. In some examples, the radial location of the first pressure port 102A can be defined with respect to the second pressure port 102B. For instance, the radial location of the first pressure port 102A can include an angular difference between 0 and 360 degrees as measured from the second pressure port 102B. In an example, the pressure port 102A can be positioned to oppose the pressure port 102B. For instance, the pressure port 102A can have a radial location 180 degrees from the pressure port 102B. In some examples, the pressure ports 102A and 102B can have the same axial location and different radial locations or different axial locations and the same radial location.

The pressure sensors, such as one of the pressure sensors 112A or 112B, can be fluidly coupled with the respective pressure port, such as one of pressure ports 102A or 102B. The pressure sensors can be located within the distal end 120 of the shaft 104. For instance, the pressure port 102A and the pressure port 102B can include an axial location at a distance P from the insertion tip 122. As shown in the example of FIG. 1, the pressure sensor 112A can be fluidly coupled with the first pressure port 102A and the second pressure sensor 112B can be fluidly coupled with the second pressure port 102B. Accordingly, the pressure sensor, such as one of the pressure sensors 112A and 112B, can detect the pressure at one of the respective pressure ports 102A or 102B respectively. In some examples, the catheter 101 can include a single pressure port. The respective pressure sensors, such as 112A or 112B, can be fluidly coupled to the single pressure port.

The pressure sensors, such as the pressure sensor 112A or 112B, can be configured to generate an electrical signal corresponding to a pressure at the respective pressure port. For instance, the catheter 101 can be inserted into the brain of a patient. The one or more pressure ports can be used to measure the pressure within the skull (intercranial pressure), such as the pressure of CSF within the ventricles or pressure within the brain tissue. In various examples, static pressure or dynamic pressure can be measured. The pressure sensor can include, but is not limited to, a piezo electric transducer, capacitive diaphragm, electromagnetic diaphragm, piezo resistive strain gauge, optical membrane or grating, plunger coupled with potentiometric sensor, pressure membranes consisting of carbon nanotubes, gold or silver nanoparticles, or other type of device for converting a pressure at the pressure port to an electrical signal usable by the processing unit 114. Providing a plurality of sensors, such as the sensor 112A or 112B, can provide redundancy to the pressure measurement at the distal end 120 of the catheter 101.

In a further example, where the pressure ports corresponding with the plurality of sensors are located in different (e.g., opposing orientations), such as having radial locations 180 degrees apart, the respective signals generated by the pressure sensors can be used for signal noise reduction, sensor failure detection, or other evaluation of pressure measurements, such as detecting measurement error that does not correspond with the actual intercranial pressure. For instance, in some examples, movement of the catheter 101 within the brain can be associated with an increase in pressure at one or more of the pressure ports. The increase in pressure can be correlated to physical contact with the tissue of the brain, the ventricular wall, or the surface of the foramen of Monro. This increased pressure measurement may not be an accurate representation of the static pressure within the brain, such as within one or more ventricles of the brain, or the static pressure of the CSF within the brain. Providing a plurality of pressure sensors, a plurality of pressure ports, or various combinations thereof, can be used to analyze the pressure signals and measure the static pressure within the brain. For instance, the measurements from the respective pressure sensors can be compared or filtered. In an example, the processing unit can determine if one or more of the electrical signals from the respective pressure sensors corresponds to a pressure that is inside of a tolerance range. The processing unit 114 can provide the output signal based on one or more electrical signals that are within the tolerance range. For instance, the tolerance range can include electrical signals that correspond to pressures in the range of normal to elevated intercranial gage pressures, such as 7 mmHg, 25 mmHg, or any value therebetween. In some examples, the tolerance range can be based on a Fourier space, wavelet space, a combination thereof, or other mathematical basis. Accordingly, a defect in the pressure measurement can be detected and the processing unit can use the electrical signal from different pressure sensors for measurement. Removal and re-insertion of the catheter 101 can be avoided along with the increase in trauma and time associated therewith.

In an example, the pressure port, such as pressure port 102A or pressure port 102B can include a membrane. A fluid can be located within a channel between the membrane and the pressure sensor. The fluid can communicate the pressure at the pressure port to the pressure sensor. Accordingly, in some examples, the one or more pressure sensors, such as the pressure sensor 112A or 112B, can be located externally to the catheter 101.

The processing unit 114 can be communicatively coupled to the one or more pressure sensors, such as the pressure sensor 112A or 112B. In the example of FIG. 1, the pressure sensor 112A and 112B are hardwired to the processing unit 114. In other examples, the processing unit 114 can be wirelessly coupled to the pressure sensors or other electronic components (e.g., electrodes, electrical contacts, sensors) of the extraventricular drain system 100. The processing unit 114 can include, but is not limited to, a central processing unit (CPU), graphics processing unit (GPU), application-specific integrated circuit (ASIC), field programmable gate array (FPGA), or other type of integrated circuit. In some examples, the processing unit 114 can be entirely hardware implemented, for instance, not including software. The processing unit 114 can further include one or more of an analog to digital converter, high pass filter, nonlinear adaptive filter (e.g., denoising filter), a Kalman filter, classification engine, neural network, correlation integral calculator, artifact detector, wavefront detector, comparator, confidence calculator, tracking module, or memory module, among other things.

The output device 116 can be communicatively coupled with the processing unit 114. In an example, the output device can be integrated with the processing unit 114. The output device can receive the one or more electrical signals (e.g., electrical signals corresponding to a physiological parameter detected by a sensor or an electrophysiological signal) from the processing unit 114 and provide an output. For instance, the processing unit 114 can provide analysis or adjustment (or a combination thereof) of the electrical signals received from the respective pressure sensors and transmit an output signal to the output device 116. The output device 116 can include, but is not limited to, a graphical display, light module, external drainage and monitoring system, medical monitor (physiological monitor), audio speaker, buzzer, vibration motor, wired or wireless transceiver, or other device configured to communicate pressure measurement results to a user or other electronic device.

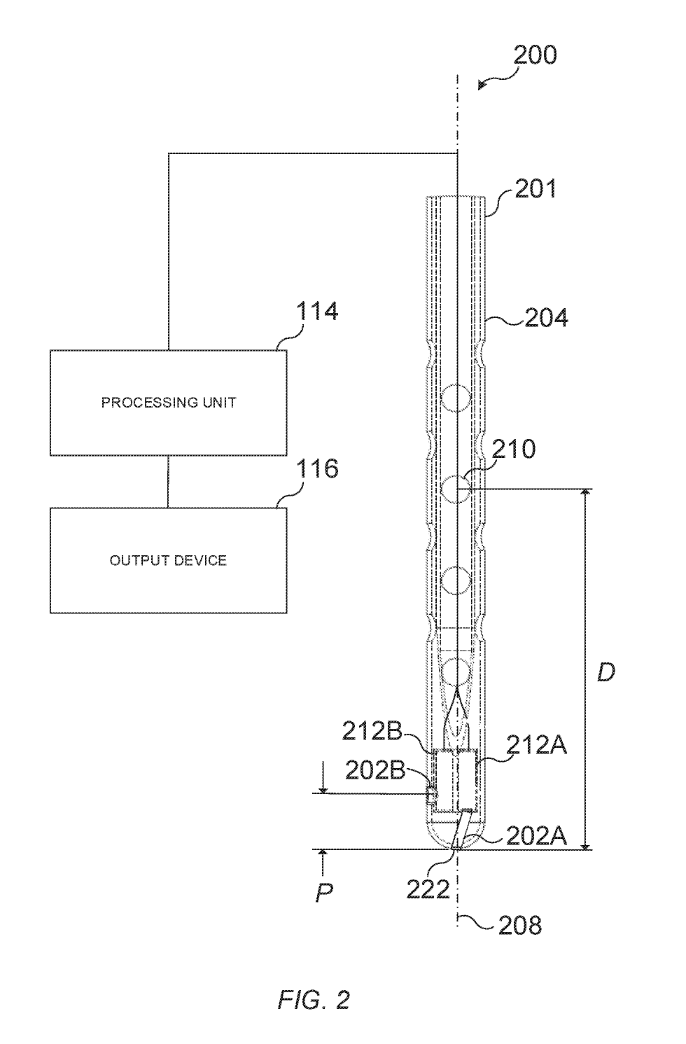

FIG. 2 is an example of an extraventricular drain system 200 including a shaft 204, an insertion tip 222, a pressure port 202A, a pressure port 202B, a pressure sensor 212A, and a pressure sensor 212B. In the example of FIG. 2, the pressure port 202A has a different radial location and axial location than the pressure port 202B. For instance, the pressure port 202A can be located on the insertion tip 122, such as along a longitudinal axis 208 of the shaft 204. The pressure port 202B can include an axial location at a distance P from the insertion tip and a radial location on the shaft 104, such as a radial location of 0, 90, 180, 270 degrees or any radial location therebetween. A plurality of pressure sensors, such as the pressure sensor 212A or 212B, can be fluidly coupled with the pressure ports. In various examples, the pressure sensors can be fluidly coupled with the pressure port 202A or 202B respectively or the pressure sensors can be fluidly coupled to both pressure ports 202A, 202B. As shown in FIG. 2, the pressure sensor 112A can be fluidly coupled with the pressure port 202A and the pressure sensor 112B can be fluidly coupled with the pressure port 202B. In an example where the location of the pressure port 202A has a different radial and axial location than the pressure port 202B, different static or dynamic pressures can be detected by the respective pressure sensors. For example, a dynamic pressure can be detected along the longitudinal axis 108 at pressure port 202A when the catheter 101 is moved along the longitudinal axis 108 (e.g., during insertion). The static pressure can be measured at the pressure port 202B. The extraventricular drain system 200, catheter 201, shaft 204, insertion tip 222, pressure port 202A, pressure port 202B, pressure sensor 212A, pressure sensor 212B, longitudinal axis 208, and drain 210 can include aspects described in conjunction with the examples of the extraventricular drain system 100, catheter 101, shaft 104, insertion tip 122, pressure port 102A, pressure port 102B, pressure sensor 112A, pressure sensor 112B, longitudinal axis 108, and drain 110.

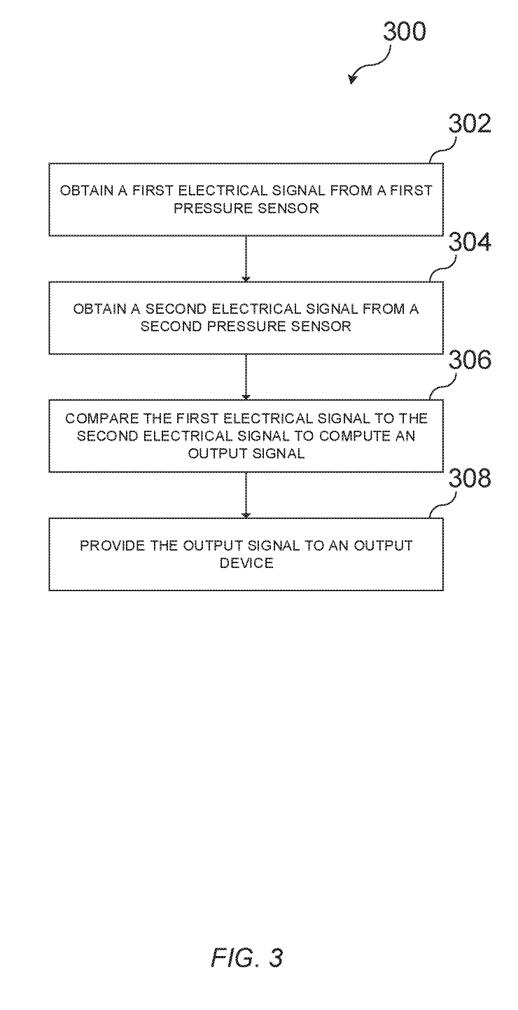

FIG. 3 is a block diagram of an example of a technique 300 for configuring a processing unit, such as the processing unit 114, to measure intercranial pressure. The processing unit 114 can include a storage device (e.g., non-transitory computer memory or computer readable medium). The storage device can include instructions for execution by the processing unit 114. The instructions can configure the processing unit to perform the operations 302-308.

At 302, a first electrical signal can be obtained from a first pressure sensor. The first pressure sensor (e.g., pressure sensor 112A) can be fluidly coupled to a first pressure port, such as pressure port 102A, as previously described.

At 304, a second electrical signal can be obtained from a second pressure sensor, such as pressure sensor 112B. The second pressure sensor can be fluidly coupled to a second pressure port, such as pressure port 102B. In various examples, the first electrical signal or the second electrical signal can be obtained using a wired or wireless connection as previously discussed.

At 306, the first electrical signal can be compared to the second electrical signal to compute an output signal. The processing unit can be configured to reduce signal noise from the output signal that is detected in the first electrical signal and the second electrical signal. In a further example, the processing unit can be configured to determine if the first electrical signal or the second electrical signal is inside a tolerance range. In an example, the tolerance range can be a person's normal (e.g., healthy) intercranial gage pressure, such as 7 mmHg, 15 mm Hg, or any value therebetween. In other examples, the tolerance range can include elevated intercranial gage pressures, such as 20 mmHg, 25 mmHg, or any value therebetween. The output signal can be based on electrical signals that are within the tolerance range. In a further example, the tolerance range can be a range of intercranial pressures that may be present in a healthy or injured person, such as a person with a traumatic brain injury. Pressures below possible intercranial pressures or above possible intercranial pressures can be outside of the tolerance range, for instance, atmospheric pressure, negative pressures, or higher pressures that exceed previously known intercranial pressures. In some examples, the tolerance range can be based on a Fourier space, wavelet space, a combination thereof, or other mathematical basis. In some examples, the first electrical signal can be compared to the second electrical signal based on signal decomposition, frequency, wavelet analysis, or causal inference using a measure of correlation or causality including cross-correlation, cross-coherence. Granger causality, and/or transfer entropy. Accordingly, a defect in the pressure measurement can be detected and the processing unit can use the electrical signal from different pressure sensors for measurement. Removal and re-insertion of the catheter 101 can be avoided along with the increase in trauma and time associated therewith.

The output signal can be based on the first electrical signal, the second electrical signal, or a combination thereof. For instance, the output signal can include both the first electrical signal and the second electrical signal to communicate the measured pressure at the first pressure port and the second pressure port respectively. In a further example, the output signal can include one of the first electrical signal or the second electrical signal. In some examples, the output signal can include a hybrid of the first electrical signal and the second electrical signal or a modified first electrical signal or modified second electrical signal. For instance, a noise reduction filter can be applied to the first electrical signal or the second electrical signal.

At 308, the output signal can be provided to an output device, such as the output device 116, as previously discussed. In one example, the output signal can be based on a pressure measured at the first port and a pressure measured at the second port to determine whether the first pressure sensor, second pressure sensor, or both correspond with a pressure at the distal end, such as the distal end 120 or insertion tip 222 of the shaft 104, 204. In an example, the processing unit can classify the pressure measured at the first pressure port or the pressure measured at the second pressure port to correspond with the pressure at the distal end where the pressure measured at the first pressure port or the pressure measured at the second pressure port are within the tolerance range.

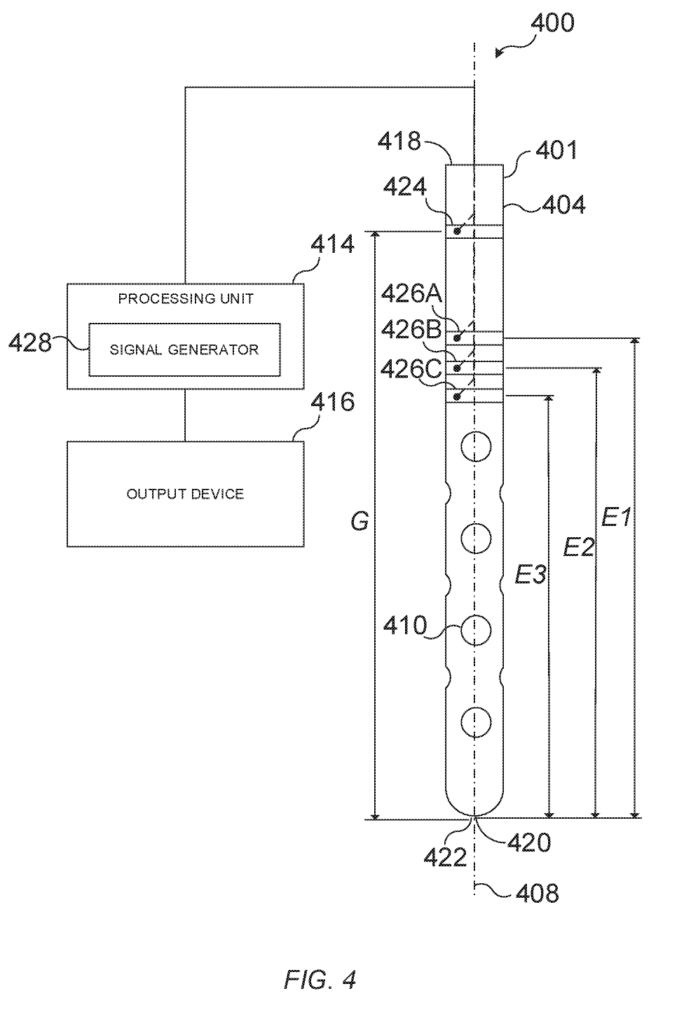

FIG. 4 depicts an example of an extraventricular drain system 400 including a reference contact 424 (e.g., a ground contact, in some examples, coupled to a grounding system) and an electrode, such as one or more electrodes 426A-C, located along a shaft 404 of a catheter 401. The catheter shaft 404 can include a proximal end 418 and a distal end 420. An insertion tip 422 can be located at the apex of the distal end 420. As shown in the example of FIG. 4, the extraventricular drain system 400 can include a processing unit 414 and an output device 416. The extraventricular drain system 400 (catheter 401 [including the shaft 404 and drain 410], processing unit 414, and the output device 416) can include aspects described in conjunction with the examples of the extraventricular drain system 100 (catheter 101 [including the shaft 104 and drain 110], processing unit 114, or output device 116).

The reference contact 424 can be located along the shaft 404 at an axial location, such as an axial location at a distance G from the distal end 420. The reference contact 424 can be communicatively coupled to the processing unit 414. For instance, the reference contact 424 can be wired or wirelessly coupled to the processing unit 414. The reference contact 424 can be electrically conductive and located along the shaft 404 to make contact with tissue or bodily fluid of a patient when inserted into the skull or brain. In the example of FIG. 4, the reference contact 424 can include a shape that surrounds (e.g., circumscribes) the periphery of the shaft 404. For instance, the reference contact 424 can have a ring shape. In other examples, the reference contact can be sized and shaped for positioning at one or more radial locations on the shaft 404, such as at 0, 90, 180, 270 degrees, or any location therebetween.

The extraventricular drain system 400 can include at least one electrode, such as one of electrodes 426A-C, to detect an electrophysiological signal and communicate a corresponding electrical signal to the processing unit 414. In the example of FIG. 4, the extraventricular drain system 400 includes three electrodes 426A-C. Electrophysiological signals can include, but are not limited to, cerebral spreading depression (CSD), synchronous neuronal activity (e.g., electrical activity corresponding to seizures), and other electrical activity within the brain. The electrode 426 can be electrically conductive and located along the shaft 404 to make contact with tissue or bodily fluid of a patient when inserted into the skull or brain. The electrophysiological signal can be received at the one or more electrodes and a corresponding electrical signal can be communicated from the electrode to the processing unit. For instance, the electrical signal can be electrically communicated to the processing unit 414 through the respective electrode, via a wire, or by using a wireless connection. In some examples, by including the electrode (e.g., one or more electrodes 426A-C) on the catheter 401, the electrophysiological signal can be measured over a longer period to provide a better understanding of CSDs, seizures, or other brain activity following TBI and allow a more accurate understanding of the physiologic and pathophysiologic factors that influence initiation of pathologic events.

In an example, at least one electrode, such as one of the electrodes 426A-C, can be electrically coupled with a signal generator 428 for providing neuromodulation treatment to the brain. The signal generator 428 (e.g., arbitrary waveform generator (AWG)) can generate a repeating or non-repeating electrical signal (e.g., via arbitrary waveform generation). In some examples, the signal generator 428 can include an amplifier or an analog to digital converter. Using at least one of the electrodes 426A-C, the signal generator 428 can provide the electrical signal for transmission to the brain. In some examples, the processing unit 414 can be communicatively coupled to the signal generator and can be configured to control the operation of the signal generator 428 according to a neuromodulation treatment (e.g., one or more parameters of a neuromodulation treatment profile). The one or more parameters of the neuromodulation profile can be stored on a storage device (e.g., computer memory) of the processing unit and can include instructions stored within the storage device for execution by the processing unit 414.

The electrode 426 can have an axial location, such as an axial location E1, E2, or E3, as depicted in the FIG. 4. The axial location can be between the reference contact 424 and the insertion tip 422. In some examples, the electrode can circumscribe the shaft 404 (can extend around the periphery or circumference of the shaft 404). For instance, the electrode can have a ring shape. In other examples, the electrode can be located partially around the shaft 404 (partially around an outer circumference of the shaft 404). The size and shape of the electrodes 426 can be calibrated to detect electrophysiological signals (e.g., corresponding to pathological states) within the brain, such as CSD, seizure activity, or other brain activities, including normal brain activity. For instance, the electrode can include a size and shape that facilitates receiving the electrophysiological signal, such as CSD or seizure activity. In some examples, if the electrode is too large, signal noise can be increased. If the electrode is too small, an incomplete electrophysiological signal may be received by the electrode.

In the example of FIG. 4, the extraventricular drain system 400 can include a plurality of electrodes 426A-C located along a longitudinal axis 408 of the shaft 404 (having respective axial locations). For instance, the first electrode 426A can have a first axial location E1, the second electrode 426B can have a second axial location E2, and the third electrode 426C can have a third axial location E3, as depicted in FIG. 4. In some examples, each of the electrodes (e.g., 426A-C) can be located between the reference contact 424 and the insertion tip 422.

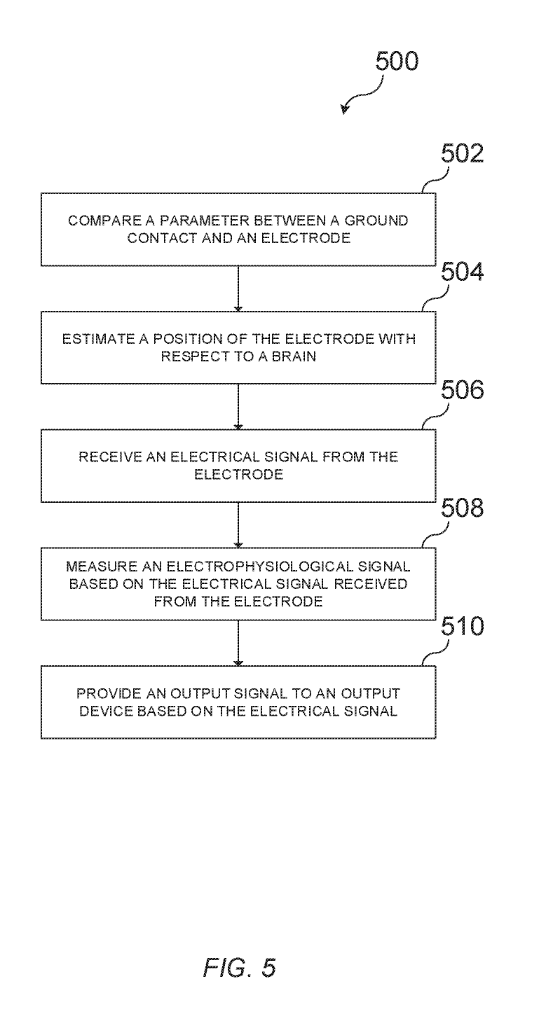

FIG. 5 is block diagram of an example of a technique 500 for measuring electrophysiological signals of the brain using an extraventricular drain, such as extraventricular drain system 400. For instance, the storage device of the processing unit 414 can include instructions that, when executed by the processing unit, configure the processing unit to perform any one of the operations 502-510.

At 502, at least one parameter between the reference contact (e.g., reference contact 424) and the electrode (e.g., 426A) can be compared. For instance, the one or more parameters can include, but are not limited to, various electrical signal characteristics, such as phase, amplitude, wavelength, frequency, or other signal characteristics that can distinguish one or more of the electrical signals from one another. In an example, the parameter for comparison can include impedance between the reference contact and the electrode. In some examples, comparing the parameter can include, but is not limited to, signal decomposition, frequency, wavelet analysis, or causal inference using a measure of correlation or causality including cross-correlation, cross-coherence, Granger causality, and/or transfer entropy.

The reference contact can be located at a first axial location along a shaft of a catheter, and the electrode is located along a second axial location of the shaft, the second axial location being between the first axial location and a distal end of the shaft. In operation, the reference contact can be positioned to contact the patient. For instance, the reference contact can be positioned to contact the scalp, skull, cranial fluid, or other anatomy of the head. In an example, the processing unit is configured to compare the parameter (e.g., impedance) between the reference contact and a plurality of electrodes (e.g., electrodes 426A-C). In some examples, the plurality of electrodes can be located along the shaft between the reference contact and the distal end, as shown in the example of FIG. 4.

At 504, a position of the electrode with respect to the brain can be estimated based on the impedance between a reference contact and an electrode. It can be desirable to measure the electrophysiological signal near the surface of the brain (surface of the cortex), for instance, to track electrophysiological activity (for example CSD, seizure, or other brain activity) along the surface of the brain. Where the extraventricular drain includes a plurality of electrodes (e.g., electrodes 426A-C), such as the extraventricular drain system 400, the processing unit can be configured to select one electrode of the plurality of electrodes for measuring the electrophysiological signal. For instance, the processing unit 414 can be configured to compare the parameter between the reference contact 424 and each of the respective electrodes, such as the electrodes 426A-C. At least one of the respective electrodes can have an impedance characteristic associated with the electrode being located closer to a surface of the cortex than the other respective electrodes. For instance, the processing unit can estimate which electrode is located within the brain and closest to the surface of the brain. In an example, the processing unit can determine if one or more of the electrodes correspond with an impedance measurement that is within a tolerance range. The tolerance range can be a range of impedance values corresponding to the impedance characteristic of an electrode within brain or within the brain and adjacent to the cortex. In some examples, the tolerance range can be based on a Fourier space, wavelet space, a combination thereof, or other mathematical basis. Accordingly, the processing unit can determine which electrode of the plurality of electrodes is positioned within the brain and closer to the cortex surface using the relative impedance measurement between the reference contact and each of the respective electrodes.

At 506, an electrical signal can be received from the electrode. The electrical signal can correspond to an electrophysiological signal of the brain. For instance, the electrophysiological signal can be received at the electrode and the corresponding electrical signal can be communicated to the processing unit through the electrode. In some examples, the electrophysiological signal can include, but is not limited to, cortical spreading depression (CSD), synchronous electrical brain activity (e.g., electrical activity corresponding to seizures), and other pathological or normal electrical activity within the brain.

At 508, the electrophysiological signal can be measured by the processing unit based on the electrical signal received from the electrode. For instance, one or more parameters of the electrical signal can be detected and recorded by the processing unit. In some examples, the one or more parameters of the electrical signal can be recorded to a storage device for retrieval at a later time.

At 510, an output signal can be provided to an output device, such as the output device 116 or 416, based on the electrical signal communicated from the electrode. In various examples, the output signal can be communicated to the output device using a wired or wireless connection. The output signal can include, but is not limited to, the electrical signal, electrophysiological signal, or an adjusted electrical signal. For instance, the output signal can include at least a portion of the electrical signal or can be based on the electrical signal, such as a filtered electrical signal.

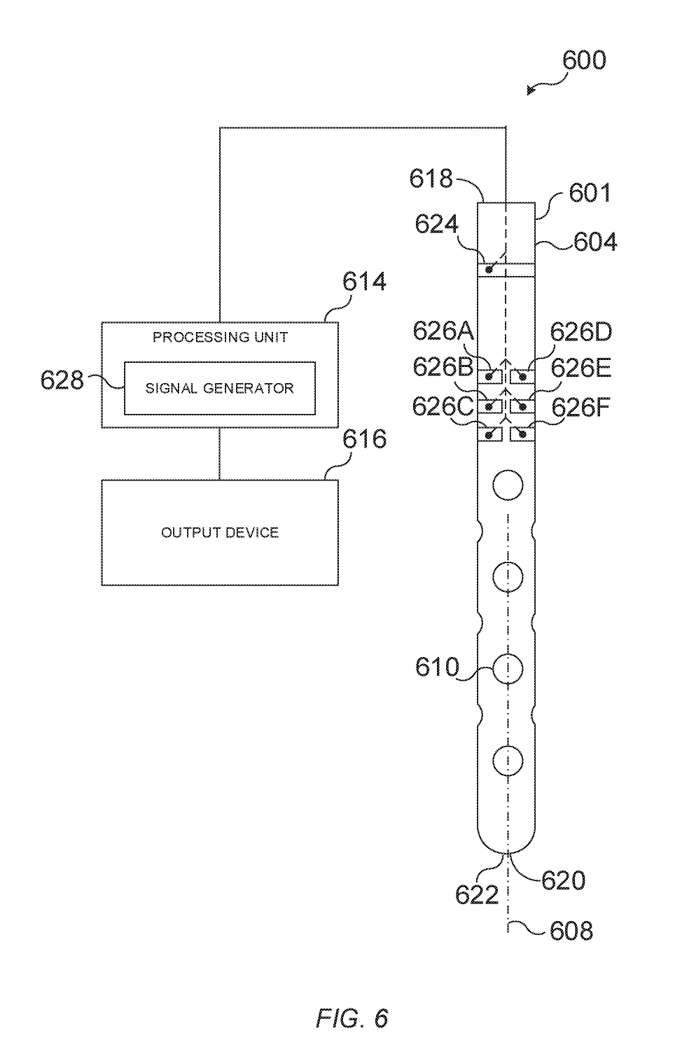

FIG. 6 is an example of an extraventricular drain system 600 including a plurality of electrodes having different radial locations along a shaft 604 of a catheter 601. For instance, the electrodes, such as electrodes 626A-C, can have different radial locations than electrodes 626D-F. As previously discussed with respect to the pressure port, the radial location can be a location on the shaft 604 at least partially transverse to a longitudinal axis 608. For instance, the radial location can be a location on the shaft 604 at a radial of the longitudinal axis 608 between 0 and 360 degrees. In an example, the first electrode can have an opposing radial location on the shaft 604 with respect to the second electrode. In various examples, the extraventricular drain system 600 can include, but is not limited to, two, three, four, or other number of electrodes having different radial locations along the shaft 604. In some examples, at least two of the electrodes can have the same longitudinal location.

As shown in the example of FIG. 6, the extraventricular drain system 600 can include a processing unit 614, signal generator 628, and output device 616, communicatively coupled to the electrodes. The extraventricular drain system 600 (catheter 601 [including the shaft 604, and drain 610, proximal end 618, distal end 620, or insertion tip 622], processing unit 614, output device 616, reference contact 624, or electrodes 626A-C) can include aspects described in conjunction with the examples of the extraventricular drain system 100 or 400 (including the shaft 104, 404; catheter 101, 401; processing unit 114, 414; output device 116, 416; or drain 110, 410), as previously described herein. In some examples, the extraventricular drain system 600 can be combined with any of the examples of the extraventricular drain systems 100, 200, or 400.

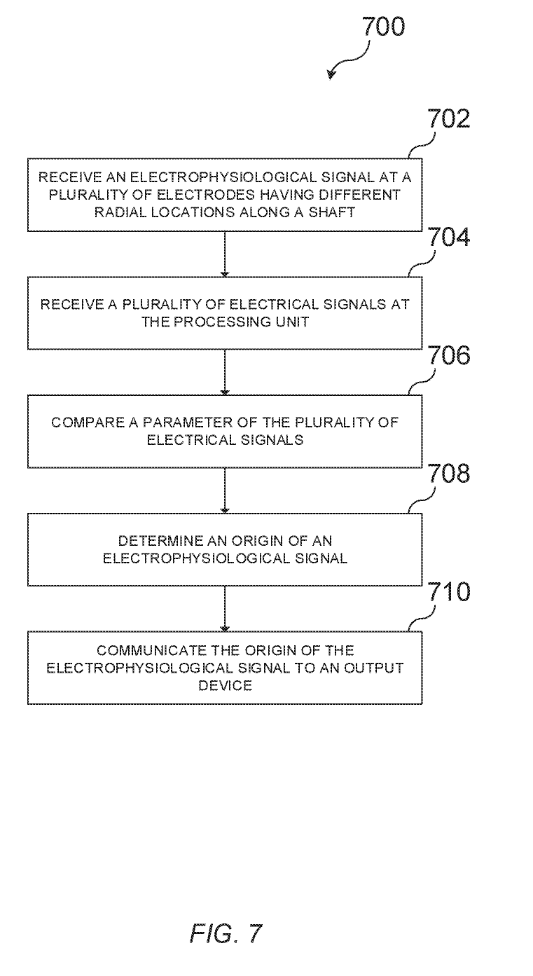

FIG. 7 is block diagram of an example of a technique 700 for determining an origin of an electrophysiological signal using the extraventricular drain system 600 for measuring an electrophysiological signal. For instance, the storage device of the processing unit 614 can include instructions that, when executed by the processing unit, configure the processing unit to perform any one of the operations 702-710.

At 702, an electrophysiological signal can be received at a plurality of electrodes having different radial locations along a shaft (e.g., shaft 604). At 704, a plurality of electrical signals can be received at the processing unit. The plurality of electrical signals can correspond to the electrophysiological signal received at the respective electrodes. For instance, a first electrical signal can be received from a first electrode, such as electrode 626A and a second electrical signal can be received from a second electrode, such as electrode 626D. The radial or longitudinal locations of the respective electrode along the shaft 604 can cause the one or more parameters of the various electrical signals, such as the first electrical signal or the second electrical signal, to be different.

At 706, at least one parameter of the plurality of electrical signals can be compared. For instance, the parameter can include, but are not limited to, various electrical signal characteristics, such as phase, amplitude, wavelength, frequency, or other signal characteristics that can distinguish one or more of the electrical signals from one another. In an example, the processing unit can detect differences among the one or more parameters of the plurality of respective electrical signals. For instance, the first electrical signal can have a phase shift with respect to the second electrical signal. In some examples, comparing the parameter can include, but is not limited to, signal decomposition, frequency, wavelet analysis, or causal inference using a measure of correlation or causality including cross-correlation, cross-coherence. Granger causality, and/or transfer entropy.

At 708, an origin of an electrophysiological signal can be determined. For instance, the processing unit can determine the origin of the electrophysiological signal based on the differences among the one or more parameters of the plurality of respective electrical signals received at the corresponding electrodes. In an example, the origin can be determined based on a correspondence between the radial location of one or more electrodes and the respective parameter of the one or more electrical signals received at the respective electrodes. For instance, the processing unit can determine the origin using a comparison of the phase between the electrophysiological signal received at a first electrode and the electrophysiological signal received at a second electrode. Additionally or alternatively, the processing unit can use amplitude, constructive or destructive interference, modeling, wavefront analysis, or any combination thereof to determine the origin of the electrophysiological signal. Determining the direction of propagation from the origin or the origin of the electrophysiological signal (e.g., CSD, seizure, or other brain activity) can be used to correlate locations within the brain that are associated with structural characteristics and pathology that gives rise to the respective electrophysiological phenomenon.

At 710, the origin of the electrophysiological signal can be communicated to an output device and presented to the user. For instance, an output signal can be provided to the output device, such as the output device 116, 416, or 616, based on the calculated origin. In various examples, the output signal can be communicated to the output device using a wired or wireless connection.

In some examples, the extraventricular drain system can include at least one pressure sensor (e.g., pressure sensor 112A or 112B), a reference contact (e.g., reference contact 424), and at least one electrode (e.g., electrode 426A-C or 326A-F). In an example including a combination of the at least one pressure sensor, a reference contact, and at least one electrode, the pressure sensors can be positioned at various axial locations on the shaft, including near the distal end as described above in reference to the drain systems 100 and 200 and as shown in FIGS. 1 and 2, and the reference contact and electrodes can be positioned at various axial locations on the shaft, including near the proximal end as described above in reference to the drain systems 400 and 600 and as shown in FIGS. 4 and 6. In an example, the reference contact can be located in proximity to the proximal end of the shaft, the pressure sensors can be located in proximity to the distal end of the shaft, and the electrodes can be located between the pressure sensors and the reference contact, but closer to the reference contact and the proximal end. The processing unit, such as the processing unit 114, 414, 624, or a combination thereof can compare electrical signals and parameters from one or more pressure sensors, reference contact, one or more electrodes, or a combination thereof. In various examples, the processing unity can measure intercranial pressure, electrophysiological signals, or combinations thereof. In some instances, the extraventricular drain system can compare intercranial pressure measurements to the measurements of electrophysiological signals for identification of corresponding physiological conditions. For example, the processing unit can identify a relationship between intercranial pressure and one or more electrophysiological signals for diagnosis, treatment, or the causation of various physiological conditions.

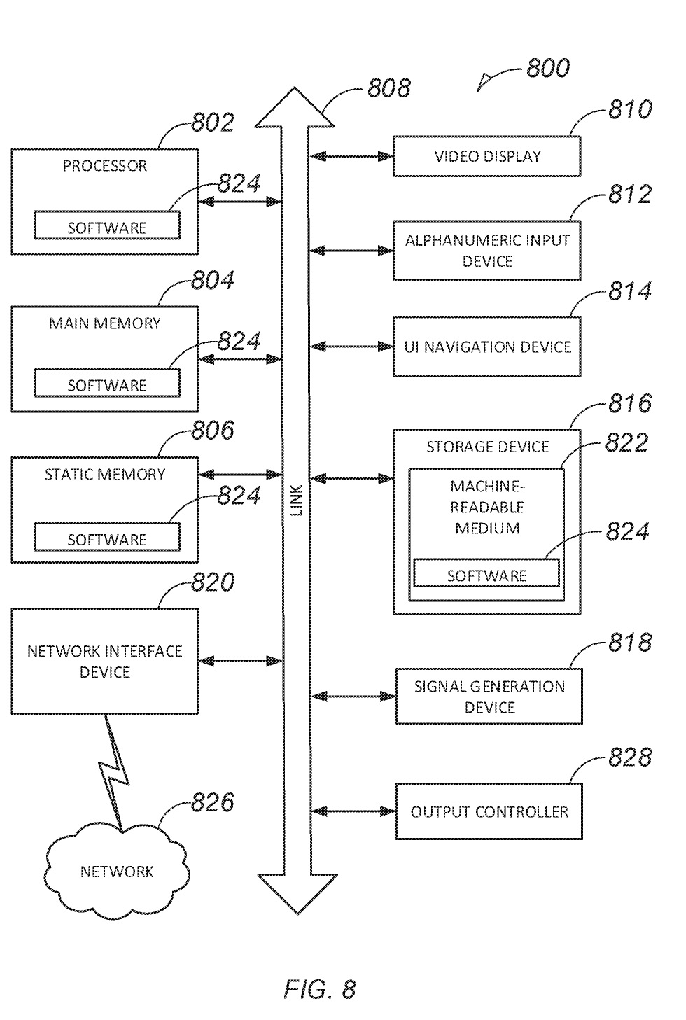

FIG. 8 is a block diagram illustrating an example machine 800 (e.g., any one of the processing units 114, 414, or 614) upon which any one or more of the techniques (e.g., methodologies) discussed herein may perform. In alternative embodiments, the machine 800 may operate as a standalone device or may be connected (e.g., networked) to other machines. In a networked deployment, the machine 800 may operate in the capacity of a server machine, a client machine, or both in server-client network environments. In an example, the machine 800 may act as a peer machine in peer-to-peer (P2P) (or other distributed) network environment. The machine 800 may be a personal computer (PC), a tablet PC, medical monitor (e.g., physiological monitor), mobile device, a web appliance, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, logic or a number of components, modules, or mechanisms. Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. In an example, the software, when executed by the underlying hardware of the module, causes the hardware to perform the specified operations.

Accordingly, the term “module” is understood to encompass a tangible entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

The machine (e.g., computer, or computer system) 800 may include a hardware processor 802 (e.g., a CPU. GPU, a hardware processor core, or any combination thereof), a main memory 804, and a static memory 806, some or all of which may communicate with each other via an interlink (e.g., bus) 808. The machine 800 may further include a display device 810, an alphanumeric input device 812 (e.g., a keyboard), and a user interface (UI) navigation device 814 (e.g., a mouse). In an example, the display device 810, input device 812 and UI navigation device 814 may be a touch screen display. The machine 800 may additionally include a mass storage device (e.g., drive unit) 816, a signal generation device 818 (e.g., a speaker), a network interface device 820, and one or more sensors 821, such as the pressure sensor 112A. 112B, accelerometer, or other sensor. The machine 800 may include an output controller 828, such as a serial (e.g., universal serial bus (USB), parallel, or other wired or wireless (e.g., infrared (IR)) connection to communicate or control one or more peripheral devices (e.g., a printer, card reader, etc.).

The mass storage device 816 may include a machine readable medium 822 on which is stored one or more sets of data structures or instructions 824 (e.g., software) embodying or utilized by any one or more of the techniques or functions described herein. The instructions 824 may also reside, completely or at least partially, within the main memory 804, within static memory 806, or within the hardware processor 802 during execution thereof by the machine 800. In an example, one or any combination of the hardware processor 802, the main memory 804, the static memory 806, or the mass storage device 816 may constitute machine readable media.

While the machine readable medium 822 is illustrated as a single medium, the term “machine readable medium” may include a single medium or multiple media (e.g., a centralized or distributed database, and/or associated caches and servers) that arranged to store the one or more instructions 824.

The term “machine readable medium” may include any medium that is capable of storing, encoding, or carrying instructions for execution by the machine 800 and that cause the machine 800 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine-readable medium comprises a machine-readable medium with a plurality of particles having resting mass. Specific examples of massed machine-readable media may include: non-volatile memory, such as semiconductor memory devices (e.g., Electrically Programmable Read-Only Memory (EPROM). Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 824 may further be transmitted or received over a communications network 826 using a transmission medium via the network interface device 820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks). Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMAX®), peer-to-peer (P2P) networks, among others. In an example, the network interface device 820 may include one or more physical jacks (e.g., Ethernet, coaxial, or phone jacks) or one or more antennas to connect to the communications network 826. In an example, the network interface device 820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 800, and includes digital or analog communications signals or other intangible medium to facilitate communication of such software.

Each of these non-limiting examples may stand on its own, or may be combined in various permutations or combinations with one or more of the other examples. The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

For example, it is to be understood that the examples of the extraventricular drain systems 100, 200, 400, or 600 can be practiced as individual systems or can be combined as various combinations thereof. In a further example, the methods 300, 500, and 700 can be practiced individually or in combination were the corresponding extraventricular drain system includes the respective structure referenced in the associated methods.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An extraventricular drain system comprising:
a catheter including:
a shaft including a proximal end and a distal end;
a lumen extended along a length of the catheter;
a drain located on the shaft, wherein the drain is fluidly coupled with the lumen;
a first pressure port located at the distal end and having a first axial location, or a first radial location, or a combination thereof, wherein the first pressure port is configured for fluid communication with a first pressure sensor; and
a second pressure port located at the distal end and having a second axial location, or a second radial location, or a combination thereof that are different than the first axial location or first radial location, wherein the second pressure port is configured for fluid communication with a second pressure sensor.

2. The extraventricular drain system of claim 1, wherein the second port is located on the shaft along the longitudinal axis.

3. The extraventricular drain system of claim 1, wherein the first pressure port and the second pressure port are located at the same axial location along the shaft, and the first radial location opposes the second radial location.

4. The extraventricular drain system of claim 1, wherein the first pressure port is fluidly coupled with the first pressure sensor and the second pressure port is fluidly coupled with the second pressure sensor.

5. The extraventricular drain system of claim 1, wherein the first pressure sensor and the second pressure sensor are configured to generate respective electrical signals corresponding to a respective first pressure at the first port and a respective second pressure at the second port.

6. The extraventricular drain system of claim 1 further comprising:
an electrode located along the shaft at a third axial location along the longitudinal axis and proximal to the first and second pressure sensors; and
a reference contact located on the shaft at a fourth axial location proximal to the electrode, wherein the electrode and the reference contact are configured for electrical communication with a processing unit.

7. An extraventricular drain system comprising:
a catheter including:
a shaft including a proximal end and a distal end and having a longitudinal axis;
a lumen extended along a length of the catheter;
a drain located on the shaft, wherein the drain is fluidly coupled with the lumen;
an electrode located along the shaft at an axial location along the longitudinal axis; and
a reference contact located on the shaft at an axial location further from the distal end than the electrode, wherein the electrode and the reference contact are configured for electrical communication with a processing unit.

8. The extraventricular drain system of claim 7, wherein the electrode is a ring shape around a periphery of the shaft.

9. The extraventricular drain system of claim 7, further comprising a plurality of electrodes, wherein each electrode is located at a different radial location along the shaft.

10. The extraventricular drain system of claim 7, further comprising a plurality of electrodes, wherein each electrode is located at a different axial location along the shaft between the distal end and the reference contact.

11. The extraventricular drain system of claim 7, wherein the processing unit is communicatively coupled to the plurality of electrodes and configured to calculate an origin of an electrophysiological signal within a brain.

12. The extraventricular drain system of claim 7, wherein the electrode is communicatively coupled to the processing unit for measuring an electrophysiological signal.

13. The extraventricular drain system of claim 7, wherein the size and shape of the electrode is calibrated for detecting cortical spreading depression or synchronous neuronal activity within a human brain.

14. The extraventricular drain system of claim 7, wherein the electrode is electrically coupled to a signal generator for providing neuromodulation to a human brain.

15. The extraventricular drain system of claim 7 further comprising:
a first pressure port located at the distal end, wherein the first pressure port is configured for fluid communication with a first pressure sensor; and
a second pressure port located at the distal end, wherein the second pressure is configured for fluid communication with a second pressure sensor.

16. The extraventricular drain system of claim 15, wherein the first pressure port is fluidly coupled with the first pressure sensor and the second pressure port is fluidly coupled with the second pressure sensor.

17. An extraventricular drain system comprising:
a processing unit including a storage device comprising instructions, and when executed by the processing unit, the instructions configure the processing unit to:
obtain a first electrical signal from a first pressure sensor, wherein the first pressure sensor is fluidly coupled to a first pressure port located at a distal end of a shaft of a catheter, the first pressure port having a first axial location, or a first radial location, or a combination thereof;
obtain a second electrical signal from a second pressure sensor, wherein the second pressure sensor is fluidly coupled to a second pressure port located at the distal end, the second pressure port having a second axial location, or a second radial location, or a combination thereof;
compare the first electrical signal to the second electrical signal to determine an output signal; and
provide the output signal to an output device.

18. The system of claim 17, wherein the processing unit is configured to perform at least one of:
reduce signal noise from the output signal that is detected in the first electrical signal and the second electrical signal, and
determine if the first or the second electrical signals are inside a tolerance range, and provide an output signal based on electrical signals that are within the tolerance range.

19. The system of claim 17, wherein the processing unit is configured to compare a parameter between a reference contact and an electrode, wherein the reference contact is located along the shaft near a proximal end of the shaft, and the electrode is located along the shaft at an axial location between the reference contact and the first and second pressure sensors.

20. The system of claim 19, wherein the electrode includes a plurality of electrodes located along the shaft between the reference contact and the pressure sensors.
